(19) United States
(12) Patent Application Publication
(10) Pub. No.: US 2024/0255501 A1
(43) Pub. Date: Aug. 1, 2024

(54) METHOD FOR THE FABRICATION OF A FLUID FLOW REGULATING PAD FOR A LATERAL FLOW IMMUNOASSAY AND CORRESPONDING LATERAL FLOW IMMUNOASSAY

(71) Applicant: CSEM Centre Suisse d'Electronique et de Microtechnique SA - Recherche et Développement, Neuchâtel (CH)

(72) Inventors: Ye TANG, Landquart (CH); Hui CHAI GAO, Neuchâtel (CH); Silvia GENERELLI, Chur (CH)

(21) Appl. No.: 18/006,645

(22) PCT Filed: Jul. 24, 2020
(86) PCT No.: PCT/EP2020/070959

§ 371 Date: Jan. 24, 2023

(51) Int. Cl. G01N 33/543; C08B 15/04; C08L 1/02

(52) U.S. Cl. CPC G01N 33/54393; C08B 15/04; C08L 1/02; G01N 33/54388

(57) ABSTRACT

A method is disclosed for the fabrication of a lateral flow test device including a nanocellulose aerogel pad obtained by implementation of steps consisting in: a) providing a hydrogel containing nanocellulose fibers, preferably carboxylic nanocellulose fibers; b) conducting a chemical crosslinking of said carboxylic nanocellulose fibers; c) conducting a lyophilisation of the hydrogel containing the crosslinked carboxylic nanocellulose fibers so as to define a nanocellulose aerogel; and d) compacting and shaping a predefined amount of said nanocellulose aerogel so as to define the nanocellulose aerogel pad.

### CROSS-REFERENCES TO RELATED APPLICATIONS

This application is the U.S. national phase of International Application No. PCT/EP2020/070959 filed Jul. 24, 2020 the entire contents of which is hereby incorporated by reference.

### BACKGROUND OF THE INVENTION

### Field of the Invention

The invention concerns a method for the fabrication of a lateral flow test or assay device intended to detect the presence of at least one predefined chemical, biological or biochemical entity in a sample and including a fluid flow regulating pad.

Generally, a lateral flow test device comprises, following the flow direction of the sample on the test device, a sample dropping area, a conjugate area intended to include at least one free labelled entity, which is optically or magnetically detectable and is adapted to react exclusively with the predefined chemical, biological or biochemical entity so as to create a combined entity, and a detection area including at least one test line intended to bear:
either a first type detection entity adapted to react exclusively with the combined entity in order to immobilize it in the detection area,
or a second type detection entity adapted to react exclusively with the free labelled entity in order to immobilize it in the detection area.

The invention concerns a lateral flow test device of this type, including a fluid flow regulating pad, obtained by implementation of the above-mentioned fabrication method.

### Description of the Related Art

Many lateral flow assays are already known in the state of the art, in particular lateral flow immunoassays.

Indeed, lateral flow immunoassays (LFIA), also known as lateral flow immunochromatographic assays, are simple cellulose-based devices intended to detect the presence of a target analyte in a liquid sample without the need for specialized and costly equipment. LFIA is a fast point-of-care tool (POCT), which is widely applied in diagnostics for the early detection of a wide set of clinically relevant biomarkers.

However, one of the major limitations of LFIA is that it only provides qualitative and/or semi-quantitative analysis and a relatively low sensitivity. This is caused by the small sample volume and the short reaction time between the detection antibody and the analytes of interest.

LFIA is a simple diagnostic device based on the chromatography-like migration of a labelled analyte through multiple membranes, its analytical sensitivity is highly dependent on the reaction time, or incubation time, between target and AuNP-conjugate. However, LFIA usually have relative short incubation times especially in the area where the antibodies and the antigen can be in contact with each other, and here are not many options to modify the incubation time except increasing the length of the nitrocellulose strip. Several materials, e.g. polydimethylsiloxane (PDMS) paper, sponge and hydrogel-paper hybrid material have been integrated into conventional LFIAs as a shunt for improving LFIA analytical sensitivity through increasing the reaction time of the fluid because of the inverse relationship between the LFIA analytical sensitivity and the reaction time.

For instance, U.S. Pat. No. 8,399,261B2 describes a classical lateral flow test system together with methods for its use in the detection of one or more analytes. Another example is disclosed in publication US20110117636A1, in which a lateral flow immunoassay device is disclosed for qualitative or quantitative analysis of an analyte of interest in a whole blood sample with improved assessment speed and accuracy. A detection limit of 0.5 ng/ml is reported there.

A strategy of incorporating a paper-based (glass fibre) shunt and a PDMS barrier into the strip was demonstrated to achieve optimum fluidic delays for lateral flow assay (LFA) signal enhancement, resulting in 10-fold signal enhancement over unmodified LFA by Choi, J. R. et al (Analytical Chemistry, (88) 2016). PDMS was selected, because it is inexpensive, inert, non-toxic and heat resistant. However, PDMS is plastic based and not hydrophilic. The system needs to integrate two sensitivity enhancement techniques, i.e. paper-based (glass fibre) shunt pad and a PDMS barrier into the conventional LFA strip. The reaction time has been increased from 40-80 seconds, therefore 10-fold LFA analytical sensitivity improvement in nucleic acid testing has been achieved, reported by R. Tang (Scientific Reports, (7) 2017). Another method was developed for using a “stacking pad” configuration that adds an additional membrane between the conjugation pad and test pad to the conventional AuNP-based LFIA format (Scientific Reports, (8) 2018). The incorporation of a similar “stacking pad” in a membrane-based platform (including polyester, cellulose, and glass fibre) was demonstrated to extend the binding interaction of antigens and antibodies by increasing the reaction time by 15 seconds.

Apart from this, researchers also reported to use gold nanoparticles (AuNPs) as labelling carriers in combination with the enzymatic activity of the horseradish peroxidase (HRP) in order to achieve an improved optical lateral flow immunoassay performance (C. Parolo et al Biosensors and Bioelectronics 2013, Enhanced lateral flow immunoassay using gold nanoparticles loaded with enzymes). The improved detect limit was 0.2 ng/mL, but the system needs an additional enzymatic substrate.

Traditional lateral flow tests rely on visual assessment and qualitative conclusion, which limit the objectivity and information output of the assays. There are few publications and patents in the field relevant to the possibilities of quantitative analysis of lateral flow immunoassay with smartphone. Publication US20150359458A1 relates to a method for obtaining a point-of-care selected quantitative indicia of an analyte on a test strip using a smartphone involving imaging a test strip on which a colorimetric reaction of a target sample has occurred due to test strip illumination by the smartphone. Similarly, K. H. Foysal et al realized the analyte quantity detection from lateral flow assay using a smartphone (Sensors, 19 (21), 2019).

On the basis of what precedes, it appears that a need still exists for a testing device which would permit to improve assay sensitivity and enlarge the dynamic range of measurement in the field of lateral flow immunoassays and, at the same time, which would be practically applicable thanks to the use of highly biocompatible materials.

The Applicant identified nanocellulose as a possible candidate to be used in lateral flow testing devices. For example, nitrocellulose membrane is widely used in paper-based microfluidic devices (like lateral flow devices), and nanocellulose, in the form of fibre or crystal, is quite often used for carrying out a hydrophilic and biocompatible coating of the microfluidic device. Indeed, nanocellulose is an abundant, renewable, and biocompatible nanomaterial that combines a low density, high strength and flexibility with chemical inertness and possibility to modify the surface chemistry. Because of their strongly interacting hydroxyl groups, cellulose materials have a strong tendency to self-associate and form an extended network via both intramolecular and intermolecular hydrogen bonds.

Apart from the above-mentioned applications of the nanocellulose fibres or crystals, nanocellulose-based foams and aerogels have been used in applications such as food packaging, coatings, biomedical, and printed electronics devices. More specifically, nanocellulose aerogels are well-known ultra-light weight materials with exceptionally high porosity within their network structure, and are typically implemented in water purification, air filter or fire-retardant applications.

For instance, publication EP2265760A1 discloses a method for providing a nanocellulose involving modifying cellulose fibres. The method includes a first modification of the cellulose material, where the cellulose fibres are treated with an aqueous electrolyte-containing solution of an amphoteric cellulose derivative, such as carboxylic nanocellulose fibre (Tempo-CNF), which is usually used as precursors for the preparation of nanocellulose aerogel.

Another example is disclosed in publication US20190309144A1 providing a method for preparing an aerogel or a foam using nanocellulose, the method comprising forming a reaction mixture comprising a cellulose nanofibril gel, and one or more crosslinking agents under conditions sufficient to crosslink the gel, and then exchanging the solvents to form an aerogel or foam.

The above-described approaches include modification of physical and chemical properties to simultaneously improve absorption capacity, mechanical properties and hydrophobicity of CNF aerogels and cellulose submicron fibre aerogels.

However, nanocellulose aerogels have been developed for other applications than LFIA and, as such, do not possess all required properties to function properly in a sample flow regulating area.

Consequently, these known nanocellulose aerogels are not suitable to be incorporated as such into a LFIA to control the corresponding flow rate and improve its analytical sensitivity. Hence, the need still exists for a testing device as mentioned above, in particular, in which highly biocompatible materials are used.

### SUMMARY OF THE INVENTION

An aim of the invention is to propose a fabrication method for a lateral flow testing device including a fluidic flow delaying or regulating pad, and allowing the lateral flow testing device to fulfil the above-mentioned requirements in terms of ease of use, selectivity, sensitivity, dynamic range, and which would further be practically applicable thanks to the use of highly biocompatible materials.

More specifically, the invention relates to a method for the fabrication of a lateral flow test device intended to detect the presence of at least one predefined chemical, biological or biochemical entity in a sample, comprising the steps consisting in providing, following the flow direction of the sample on the test device:
a sample pad,
a conjugate pad intended to include at least one free labelled entity, which is optically or magnetically detectable and is adapted to react exclusively with said at least one predefined chemical, biological or biochemical entity so as to create a combined entity, and
a working membrane including at least one test line intended to bear:
   either a first type detection entity adapted to react exclusively with said combined entity in order to immobilize it on said working membrane,
   or a second type detection entity adapted to react exclusively with said at least one free labelled entity in order to immobilize it on said working membrane,
the method comprising a further step of integrating a fluid flow regulating pad in the lateral flow test device, wherein said fluid flow regulating pad is a nanocellulose aerogel pad manufactured by implementing steps consisting in:
a) providing a hydrogel containing nanocellulose fibres, preferably carboxylic nanocellulose fibres,
b) conducting a chemical crosslinking of the carboxylic nanocellulose fibres,
c) conducting a lyophilisation of the hydrogel containing the crosslinked carboxylic nanocellulose fibres so as to define a nanocellulose aerogel, and
d) compacting and shaping a predefined amount of the nanocellulose aerogel so as to define the nanocellulose aerogel pad.

Thanks to the above features, the fabrication method according to the present invention allows the nanocellulose aerogel to have the right surface state to minimize non-specific adsorption from molecules and increase its hydrophilicity.

On a general basis, it might be advantageous to provide carboxylic nanocellulose fibres which are of the Tempo-CNF type in step a) of the fabrication method according to the invention.

Further, step b) of the fabrication method may thus include a preliminary operation consisting in stirring a hydrogel solution containing between 0.5 and 5% in weight of Tempo-CNF (or CNF) for 30 to 60 mins at a temperature comprised between 20 and 30° C. In that case, the hydrogel solution might be stirred at a stirring rate comprised between 1000 and 3000 rpm.

Moreover, step b) preferably includes additional operations consisting in
adding between x/2000 and x/500 g of 1,2,3,4-Butanetetracarboxylic acid (BTCA) powder and between x/20000 and x/5000 g of sodium hydrosulphite (Na₂S₂O₄) powder to x g of the stirred Tempo-CNF (or CNF) hydrogel solution, and stirring the corresponding solution during at least 6 hours at a temperature comprised between 20 and 30° C.

Generally, step c) preferably includes operations consisting in
pouring the hydrogel solution containing crosslinked carboxylic nanocellulose fibres in a container such that the hydrogel solution has a final height in the container comprised between 0.5 and 10 mm,
keeping the container at a temperature comprised between 10 and 30° C. for 10 to 60 mins,
storing the container at a temperature comprised between −30 and −10° C. during at least 6 hours, and
freeze drying the hydrogel solution containing crosslinked carboxylic nanocellulose fibres at a temperature comprised between −65 and −50° C. during at least 20 hours by lyophilizing.

According to another preferred embodiment, step d) of the fabrication method may consist in applying a weight comprised between 0.5 and 10 kg on the nanocellulose aerogel during at least 10 mins to shape a nanocellulose aerogel pad having a thickness approximately comprised between 0.1 and 2 mm.

Generally, the fabrication method may further include an operation of passivation of at least part of the surface of the nanocellulose aerogel pad.

Generally, the nanocellulose aerogel pad may preferably be arranged, in the lateral flow test device, so as to contact, on the one hand, the conjugate pad and, on the other hand, the working membrane.

An additional aim of the present invention is to provide a lateral flow test device intended to detect the presence of at least one predefined chemical, biological or biochemical entity in a sample,
obtained by implementation of the above-mentioned fabrication method.

According to a preferred embodiment of the test device, the nanocellulose aerogel pad might be arranged so as to contact, on the one hand, the conjugate pad and, on the other hand, the working membrane. Thus, the flowing speed of the sample between the conjugate pad and the working membrane can be regulated, depending on the conditions of the corresponding test, in particular on the nature of the entity to be detected and on the nature of the free labelled entity with which it is intended to react for the completion of the test.

According to a preferred embodiment, the nanocellulose aerogel pad may have a thickness comprised between 0.1 and 2 mm, preferably between 0.2 and 1.0 mm, and a length comprised between 1 and 8 mm, preferably between 2 and 6 mm.

Generally, when the device is intended to detect at least one predefined antibody, the free labelled entity may comprise a first anti-antibody adapted to react with the predefined antibody to create the combined entity.

In that case, and when the test line bears a first type detection entity adapted to react exclusively with the combined entity, the first type detection entity might be a second predefined anti-antibody.

In alternative, when the test line bears a second type detection entity adapted to react exclusively with the free labelled entity, the second type detection entity might be a second predefined antibody. The second predefined antibody might correspond to the antibody to be detected for instance.

In general, the free labelled entity may advantageously contain one or more of the entities belonging to the group consisting in gold, a latex, a fluorophore, a ferromagnetic or paramagnetic entity.

According to a preferred embodiment:
the sample pad may preferably be made of cellulose fibre,
the conjugate pad may preferably be made of glass fibre, and
the working membrane may preferably be made of nitrocellulose.

### BRIEF DESCRIPTION OF THE DRAWINGS

Further details of the invention will appear more clearly upon reading the description below, in connection with the following figures which illustrate:

FIG. 1: schematic illustration of the general construction of a test device according to a preferred embodiment of the present invention;

FIG. 2: schematic illustration of the implementation of a test based on the use of a test device having a general construction as illustrated in FIG. 1;

FIG. 3: schematic illustration of the implementation of a specific step of a method for manufacturing a test device as illustrated in FIG. 1;

FIGS. 4a-4b: photos comparing the results of tests carried out with, on the one hand, test devices according to the present invention and, on the other hand, test devices according to prior art,

FIGS. 5a and 5b: Scanning electron microscopy (SEM) pictures of the surface of a nanocellulose aerogel pad according to the invention, at two different magnifications, and

FIG. 6: schematic diagram relating to the migration speed of a sample on different types of LFIAs according to the present invention or to the prior art.

FIGS. 7a-7b: schematic diagrams relating to the results of tests carried out with, on the one hand, test devices according to the present invention and, on the other hand, test devices according to prior art.

### DESCRIPTION OF THE PREFERRED EMBODIMENTS

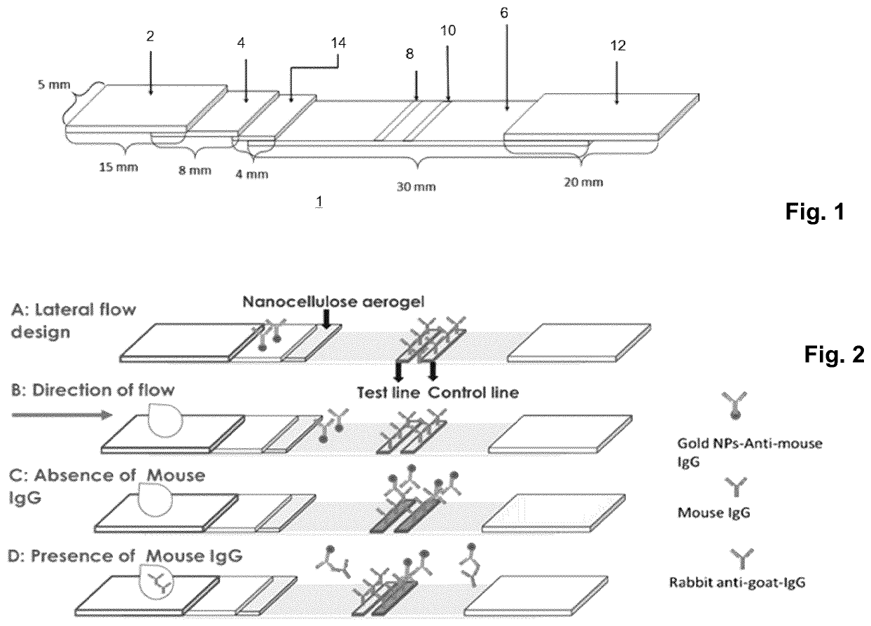

FIG. 1 is a schematic illustration of the general construction of a test device according to a preferred embodiment of the present invention, more precisely of a lateral flow immunoassay device 1 incorporating a nanocellulose aerogel pad and obtained by implementation of the fabrication method according to the present invention.

Like typical lateral flow immunoassays, the test device 1 according to the present invention comprises a sample pad 2, defining a sample dropping area from which a dropped sample is intended to start flowing on the device 1, the latter being intended to assess whether at least one predefined chemical, biological or biochemical entity is present in the sample.

Lateral flow immunoassays adapted to detect the presence of more than one predefined entity are also known, and the present invention is not limited to the detection of one entity only. The one skilled in the art will be able to adapt the present teaching to implement a test device for the simultaneous detection of more than one predefined entity without any particular difficulty and without going beyond the scope of the present invention. This includes the application of POCT for biomarkers detection in body fluids (blood, serum, saliva, urine, tear and so on) by direct immunoassay, sandwiches immunoassay or competitive immunoassay, detection of pollutants (pesticides, herbicides) in the environmental monitoring, detection of toxins, antibiotic residues, pesticides residues for food quality control.

The device 1 further comprises, following the flow direction of the sample, a conjugate pad 4, defining a conjugate area intended to include at least one free labelled entity, which is optically or magnetically detectable and is adapted to react exclusively with the predefined chemical, biological or biochemical entity to be detected so as to create a predefined combined entity.

In a well-known manner, more than one free labelled entity can be provided when more than one predefined entity has to be detected in the sample.

In a known manner, the free labelled entity may advantageously contain one or more of the entities belonging to the group consisting in gold, a latex, a fluorophore, a ferromagnetic or paramagnetic entity, so as to allow its detection with an optical or a magnetic sensor.

The device 1 further comprises a working membrane 6 defining a detection area including at least one test line 8 which might be configured in two different ways, according to two different approaches.

On the one hand, the test line 8 might bear a first type detection entity adapted to react exclusively with the combined entity in order to immobilize it in the detection area. This approach is sometimes called the “sandwich” approach as the predefined entity to be detected is able to react both with the free labelled entity in a first step, and with the first type detection entity then, in a second step, in order to immobilize the combined entity on the test line 8.

Thus, if the predefined entity to be detected is present in the sample, the combined entity will form and will be able to react with the first type detection entity. The aptitude of the free labelled entity to be optically or magnetically detected will allow a detection of the combined entity on the test line 8, to assess whether the predefined entity to be detected is present or not in the sample.

On the other hand, the test line 8 might bear a second type detection entity adapted to react exclusively with the free labelled entity in order to immobilize it in the detection area. This approach is sometimes called the “competitive” approach as the more the sample contains some predefined entity to be detected, the less some free labelled entity will remain available to be immobilized on the test line 8.

Thus, if the predefined entity to be detected is not present in the sample, all of the free labelled entity will remain available to be immobilized on the test line 8 by reaction with the second type detection entity, which can be detected thanks to the aptitude of the free labelled entity to be optically or magnetically detected. On the contrary, if the predefined entity to be detected is present in the sample, less of the free labelled entity is available to be immobilized on the test line 8, which can also be detected thanks to the aptitude of the free labelled entity to be detected.

Generally, lateral flow immunoassays include an optional control line 10 located after the test line 8, following the flow direction of the sample. The control line 10 typically bears a predefined control entity suitable to react with the free labelled entity to immobilize the latter on the control line 10, so it can be checked whether the sample flow has worked properly, by transferring the free labelled entity from the conjugate pad 4 to the control line 10 (so, at least, past the test line 8). Several control entities can be provided, on one or more control lines, when more than one predefined entity is to be detected. In the competitive approach, the control line 10 might bear a predefined control entity which is adapted to react with both the free labelled entity (in case the sample to be assessed does not contain the predefined entity to be detected) and the combined entity (in case the sample to be assessed contains the predefined entity to be detected, implying that there could possibly remain no free labelled entity when the sample flow reaches the control line 10).

A wicking pad 12 is typically provided then to receive the sample and allow the working membrane 6 to dry.

On a general basis, the sample pad 2 is typically made of cellulose fibre, while the conjugate pad 4 is made of glass fibre, the working membrane 6 is made of nitrocellulose and the wicking pad 12 is also made of cellulose fibre. However, the one skilled in the art will be able to adapt the nature of these pads as a function of his specific needs without going beyond the scope of the present invention.

Generally, when the predefined entity to be detected is an antibody, the free labelled entity may comprise a first anti-antibody adapted to react with the antibody to be detected to create the combined entity.

Then, on the one hand, when the test line 8 bears a first type detection entity adapted to react exclusively with the combined entity, i.e. in the sandwich approach, the first type detection entity may advantageously be a second predefined anti-antibody.

On the other hand, when the test line 8 bears a second type detection entity adapted to react exclusively with the free labelled entity, i.e. in the competitive approach, the second type detection entity may advantageously be a second predefined antibody or, in alternative, the same antibody as the one to be detected.

Unlike the typical approach according to which the speed of tests conducted with lateral flow immunoassays should be raised, the present invention aims at lowering the speed of these tests so as to improve their sensitivity and dynamic range. Indeed, a main aim of the present invention is to lower the speed of these tests by raising the contact time between the predefined entity to be detected and the free labelled entity, so as to ensure that as much as possible of the free labelled entity or of the predefined chemical, biological or biochemical entity has reacted to create the combined entity before the sample reaches the detection area, preferably 80%, more preferably at least 90%. Thanks to this feature, precise quantitative results can be achieved through implementation of tests with the test device according to the present invention.

For that purpose, the lateral flow test device of the invention further comprises a speed regulating pad 14 defining a sample flow regulating area, arranged in such a manner that as much as possible of the free labelled entity or of the predefined chemical, biological or biochemical entity has reacted to create the combined entity before the sample reaches the detection area. Indeed, the extension of the reaction time between the biomolecules is expected to improve the binding efficiency between target analytes and detection antibody labelled with gold nanoparticles.

As already mentioned above, nanocellulose aerogels are well-known ultra-light weight materials with exceptionally high porosity within their network structure.

Thus, the speed regulating pad 14 may advantageously include a sample flowing portion made of a nanocellulose aerogel.

However, nanocellulose aerogels have been developed for other applications and as such do not possess all required properties to function properly in a sample flow regulating area. In particular, its hydrophilicity has to be improved and there must be a minimal non-specific adsorption of molecules. This is achieved by proper chemical functionalization of its surface. In addition, the pore size of the nanocellulose aerogel has to be adjusted and optimized to allow the flow of the larger entities (for example, the size of gold nanoparticles typically varies between 10 nm and 150 nm) while keeping a sufficiently slow flow rate.

During his research work, the applicant identified the nanocellulose aerogel as being a good candidate to impact the flowing speed of the sample in a lateral flow immunoassay, so as to regulate it and control the reaction time between the predefined entity to be detected and the free labelled entity. Indeed, the applicant realized that provision of a nanocellulose aerogel pad could decrease the capillary flow rate by increasing the fluidic resistance and thus extend the reaction time between the free labelled entity and the predefined entity to be detected. For this, several modifications in the fabrication and functionalization of nanocellulose aerogel had to be implemented in order to achieve the required performances. In this work, different methods of fabrication of nanocellulose aerogel were tested and used to shape nanocellulose aerogel pads, the wet stability of which was tested to identify the most mechanically stable aerogel for the preparation of LFIA. By optimizing the thickness and length of the aerogel pads, it appeared that the specific geometry of CNF aerogel provided a proper fluid resistance in the LFIA strips. Finally, to investigate the decrease of the limit of detection brought by integrating CNF aerogel in LFIA, the behaviour of LFIAs with and without CNF aerogel were compared to each other for detecting the mouse IgG as a proof of concept.

Further details about the preferred fabrication method will be provided later in the present description.

Finally, the manufacture of nanocellulose aerogel assisted lateral flow immunoassay (NA-LFIA) strips should be easy to accomplish and feasible for automatically production line. A cost efficiency commercial product could be used to fabricate the nanocellulose aerogel, then the aerogel pad could be easily inserted between the conjugate and the nitrocellulose pads of a conventional LFIA strip setup.

Regarding the typical dimensions of known lateral flow immunoassays, as indicated in a non-limiting way in FIG. 1, the applicant could identify appropriate ranges for the dimensions of the nanocellulose aerogel sample flowing portion. More particularly, the sample flowing portion may preferably have a thickness comprised between 0.1 and 2 mm, more preferably between 0.2 and 1.0 mm, and a length preferably comprised between 1 and 8 mm, more preferably between 2 and 6 mm.

The working principle of the test device 1 is schematically illustrated in FIG. 2, in a competitive approach and for a specific embodiment regarding the predefined entity to be detected which is here an antibody, mouse IgG.

Part A of FIG. 2 illustrates the initial state, the device 1 being ready for a test.

The conjugate pad 4 contains gold nanoparticle labelled goat anti-mouse IgG antibody as the free labelled entity, adapted to react with the mouse IgG to be detected, while test line 8 bears mouse IgG, adapted to react exclusively with the free gold nanoparticles of anti-mouse IgG, and control line 10 bears rabbit anti-goat-IgG adapted to react both with the free gold nanoparticles of anti-mouse IgG and with the combined entity resulting from the reaction between the mouse IgG of the sample and the free gold nanoparticle labelled goat anti-mouse IgG antibody.

Part B illustrates what happens when a sample is dropped on the sample pad 2 and the fact that the sample starts flowing in the direction indicated by the arrow, the free gold nanoparticles of anti-mouse IgG being transferred with the sample in the direction to the detection area where the test line 8 and the control line 10 are. As long as the free gold nanoparticle labelled goat anti-mouse IgG antibody do not reach the test line 8 and the control line 10, these lines keep their initial appearance, which is generally the colour of the working membrane 6, typically white.

Part C illustrates the assessment of a sample which does not contain mouse IgG leading to a negative result. Indeed, in the absence of mouse IgG, the free gold nanoparticles of anti-mouse IgG cannot react with the sample to create a combined entity and remain thus available to react with the mouse IgG borne by the test line 8 and with the rabbit anti-goat-IgG borne by the control line 10. Consequently, when the free gold nanoparticle labelled goat anti-mouse IgG antibody reach the test line 8 and, then, the control line 10, they react with the mouse IgG and with the rabbit anti-goat-IgG respectively borne by these lines which thus both change of colour due to the immobilization there of a noticeable quantity of free gold nanoparticle labelled goat anti-mouse IgG antibody.

Part D illustrates the assessment of a sample which contains mouse IgG leading to a positive result. Indeed, as soon as the sample reaches the conjugate pad 4, the mouse IgG contained in the sample starts reacting with the free gold nanoparticle labelled goat anti-mouse IgG antibody so as to create a combined entity which is not adapted to react with the mouse IgG borne by the test line 8 but is still able to react with the rabbit anti-goat-IgG borne by the control line 10. Consequently, less or no free gold nanoparticle labelled goat anti-mouse IgG antibody reach the test line 8 the appearance of which thus changes less than in the case where no mouse IgG is present in the sample. The final colour of the test line 8 is consequently unchanged or only slightly changed, with reference to its initial appearance, depending on the concentration of the sample in mouse IgG. Obviously, the lower the concentration of the sample in mouse IgG is, the more the test line 8 will exhibit a change in its coloration. The control line 10 still changes of colour as a consequence of the reaction of the rabbit anti-goat-IgG with both the remaining free gold nanoparticle labelled goat anti-mouse IgG antibody and the combined entity.

Commercially speaking two main advantages of the present invention are to have an increased sensitivity of immunoassay by a factor of ten at least and to enlarge the dynamic range up to five orders of magnitude (from 0.01 ng/ml to 100 ng/ml with IgG-Anti-IgG model system), in comparison with conventional lateral flow immunoassay (LFIA). This is achieved by integrating a porous nanocellulose aerogel pad in the LFIA, as previously mentioned, as the nanocellulose aerogel, with its high porosity network structure, could decrease the average capillary flow rate by increasing the average fluidic resistance.

As a consequence, the predefined entity to be detected and the free labelled entity respectively eluted from the sample pad 2 and from the conjugate pad 4 could accumulate and concentrate momentarily within the nanocellulose aerogel pad or speed regulating pad 14, and the reaction time between the free labelled entity and the predefined entity to be detected could be extended as well. The composition and the geometry of the nanocellulose aerogel should preferably be cautiously selected in order to provide a reproducible fluidic resistance as well as a good chemical, biochemical and mechanical stability.

On a general basis, the present invention relates to a method for the fabrication of a lateral flow test device including a nanocellulose aerogel pad suitable as just described.

More particularly, this fabrication method includes the manufacture of a nanocellulose aerogel pad by implementation of steps consisting in:
a) providing a hydrogel containing carboxylic nanocellulose fibres,
b) conducting a chemical crosslinking of the carboxylic nanocellulose fibres,
c) conducting a lyophilization of the hydrogel containing the crosslinked carboxylic nanocellulose fibres so as to define a nanocellulose aerogel,
d) compacting and shaping a predefined amount of the nanocellulose aerogel so as to define the speed regulating nanocellulose aerogel pad.

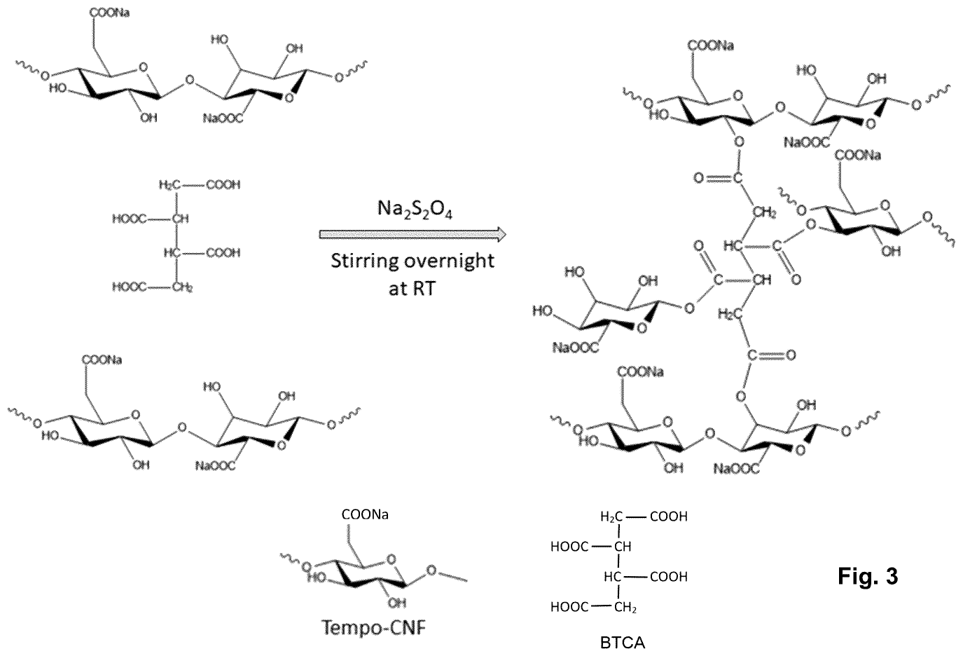

As previously mentioned, the applicant conducted many experiments in order to identify a preferred exemplary embodiment for the manufacturing method of the test device 1, as partially illustrated in FIG. 3.

Carboxylic nanocellulose fibre (Tempo-CNF) was finally selected to fabricate the aerogel by chemical crosslinking reaction, the following fabrication steps were defined after optimization, including a mechanical pre-treatment, initial concentrations of reactants, reaction times and temperatures.

More precisely, FIG. 3 schematically illustrates the chemical crosslinking operation according to the preferred embodiment of the manufacturing method of the invention. This exemplary method allows the nanocellulose aerogel to have the right surface state to minimize non-specific adsorption of molecules and increase its hydrophilicity.

This operation preferably comprises the following steps:
i) Stir 1% in weight (“1 wt %”) of Carboxylic Nanocellulose Fibre (Tempo-CNF) hydrogel mechanically for 20 min at room temperature using an overhead stirrer (2000 rpm);
ii) Add 50 mg of 1,2, 3,4-Butanetetracarboxylic acid (BTCA) powder and 5 mg of sodium hydrosulphite (Na₂S₂O₄) powder to 50 g of the 1 wt % of Carboxylic Nanocellulose Fibre (Tempo-CNF) hydrogel solution and stir permanently overnight at room temperature with a magnetic stirrer.

For example, the following details can be implemented in the crosslinking of CNF aerogel. The chemically crosslinked CNF aerogel was produced by firstly adding 2 g of TEMPO-CNF powder into 198 ml of Milli. Q water to prepare the 1 wt. % of TEMPO-CNFs suspensions. The suspension was under vigorous stirring by IKA® RW 20 stirrer (2000 rpm level) at RT for 30 mins. Then, 50 mg of 1, 2, 3, 4-butane tetracarboxylic acid (BTCA, 10 wt. % of TEMPO-CNF powder) and 5 mg of sodium hydrosulphite (SHP, 10 wt. % of BTCA) were added into the 50 mL of obtained homogeneous suspension as the chemical crosslinker. The suspension was under magnetic stirring at RT overnight to complete the formation of chemically crosslinked hydrogel via an esterification reaction between cellulose hydroxyl and BTCA carboxylic acid groups.

In order to obtain a reliable fluidic resistance and good physical, chemical and mechanical stability of nanocellulose aerogel, the thickness and freeze dry processes were also investigated and optimized.

After chemical crosslinking, 1.0-5.0 mL of nanocellulose hydrogel was poured into containers like wells with diameter of 34 mm and kept standing for 30 mins at room temperature, before being moved into a refrigerator (−20° C.) and attached to the bottom of drawer and stored overnight. Freeze drying was then carried out at −55° C. for 24 hours by lyophilizing. The large format of aerogel pads can be produced by pouring 25 mL of crosslinked nanocellulose hydrogel into a round-shape petrel dish with diameter of 90 mm, or 30 mL into a square petrel dish (90 mm×90 mm). Depending on the size of the container, the height of the hydrogel solution in the container should be approximately between 0.5 and 10 mm.

Supercritical CO₂ (scCO2) drying could also be carried out after exchanging the aqua solvent by organic solvents. Freeze drying creates micropores with a size of 50 μm-200 μm, and supercritical drying creates nanopores with a size of 2 nm-50 nm applicable to small sized entities.

Nanocellulose aerogel pads were then compacted (approximately between 5 to 10 times) with a weight of 1.0 kg for 30 min and the final thickness of the pads was 0.5 mm, which was suitable for integrating them into typical lateral flow strips.

The length and thickness of the nanocellulose aerogel pad 14 has been optimized, i.e. preferred thickness and length of the nanocellulose aerogel pad 14 are comprised between 0.1 and 2 mm, preferably comprised between 0.2 and 0.6 mm, more preferably 0.5 mm and, comprised between 1 and 8 mm, preferably comprised between 2 and 6 mm, more preferably 4 mm, respectively.

Finally, surface modification and passivation of nanocellulose aerogel pads for long-term stability can advantageously be carried out. It could be realized, for instance, by blocking the chemical active groups of nanocellulose backbone chain with amine contained polysaccharides or other inert polymers, i.e. ethanolamine can be used to neutralize the chemical active groups of nanocellulose backbone chain by adding 0.8 UL ethanolamine into 1.0 mL of nanocellulose hydrogel solution after the crosslinking reaction and before the lyophilization process. BSA is the other choice to block the chemical active groups of nanocellulose backbone chain. For instance, BSA powder can be added to nanocellulose hydrogel solution at the ratio of 0.1% (w/v) after the crosslinking reaction and incubated overnight at 4° C. before carrying out the lyophilization process.

Alternatively, CNF without carboxylic groups can be used as reactant in order to reduce the nonspecific binding from the beginning.

In order to get more convenience using LFIA with real body fluid sample, i.e. whole blood sample, an optional pre-treatment step can be provided. For instance, the surface of the nanocellulose aerogel can be modified by adding anticoagulant reagents like EDTA (1.0 mg/mL) or sodium citrate (3.0 mg/mL) in the nanocellulose hydrogel solution after the crosslinking reaction, thus avoiding coagulation. Then freezing drying can be carried out as described above.

Mechanical strength of nanocellulose aerogel can be enhanced by combining (3-Aminopropyl)triethoxysilane with the Carboxylic Nanocellulose Fibre (Tempo-CNF) hydrogel solution at a ratio from 0.3% to 0.5% (v/v).

The competitive immunoassay processes for the detection of mouse IgG with IgG-Anti-IgG model system have been optimized, as an examplary illustrative embodiment.

The response time of the assay with the nanocellulose aerogel pad 14 was about 80 seconds longer than without nanocellulose aerogel pad. Calibration curves were made by plotting the obtained value of grayscale pixel of the test line against different concentrations of mouse IgG with a fitting curve in a log-log scale. The sensitivity, dynamic range for quantification of mouse IgG were calculated.

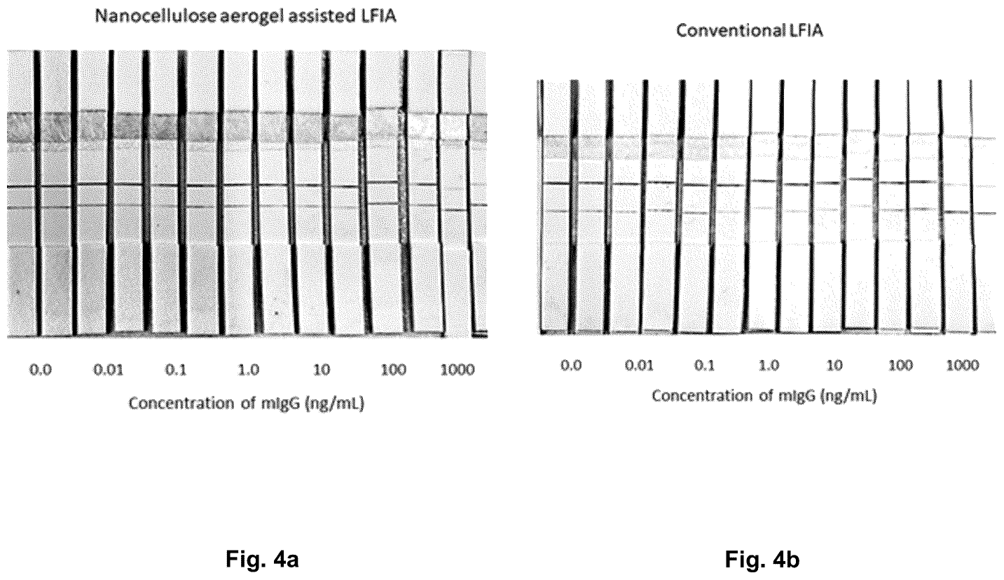

As illustrated in FIGS. 4a and 4b, the lateral flow strip with the nanocellulose aerogel assisted gives a sensitivity of 0.1 ng/ml in a linear range from 0.1 ng/mL to 100 ng/ml (FIG. 4a), while the conventional LFIA strip shows no-quantitative behaviour for detection of mouse IgG (FIG. 4b). These results confirmed that the efficiency of accumulating the analytes and extending the reaction time using nanocellulose aerogel could achieve the lower limit of detection of 0.1 ng/ml and an extended linear range with five orders of magnitude (from 0.01 ng/ml to 100 ng/ml (R=0.9696)).

The nanocellulose aerogel assisted LFIA was obtained by inserting a nanocellulose aerogel pad between the conjugate pad and the working membrane, then applied in a competitive immunoassay for colorimetric detection of mouse IgG. In the immunoassay, gold nanoparticles-anti-mouse IgG as the labelled detection antibody conjugates are dispensed on the conjugate pad. Mouse IgG and Rabbit-anti-goat IgG are immobilized on the test line and control line, respectively. In the experiment, samples containing different concentrations of mouse IgG were tested, the running buffer of assay was PBS buffer containing 0.05% of Tween 20. After 30 mins, the signal of test line from different strips were photographed with a cell phone under controlled light conditions. Subsequently, the value of grayscale pixel of the test line colour was determined with Image J software. The comparative results were obtained with the conventional LFIA strips in parallel.

In order to allow larger entities to flow across the nanocellulose aerogel while still regulating the sample flow, the pore size has to be optimized. For this, the following method was implemented.

As previously mentioned, carboxylic nanocellulose fibre (Tempo-CNF) was finally selected to fabricate the aerogel by chemical crosslinking reaction, the fabrication method steps were defined through an optimization process. The length and thickness of nanocellulose aerogel pad has been optimized, i.e. the thickness and the length of aerogel pad are 0.5 mm and 4.0 mm, respectively. However, the length and thickness of nanocellulose aerogel pad is inversely proportional to the sample flow speed, so it can be adjusted to adapt the requirement of various applications.

On the other hand, the chemical crosslinked CNF aerogel exhibits an interconnected porous structure with a pore size of aerogel between 100 and 200 μm by implementation of the step as described in FIG. 3. The pore size of aerogel can be obtained in different ranges by adjusting the initial concentration of reactants, especially for carboxylic nanocellulose fibre (Tempo-CNF) hydrogel solution.

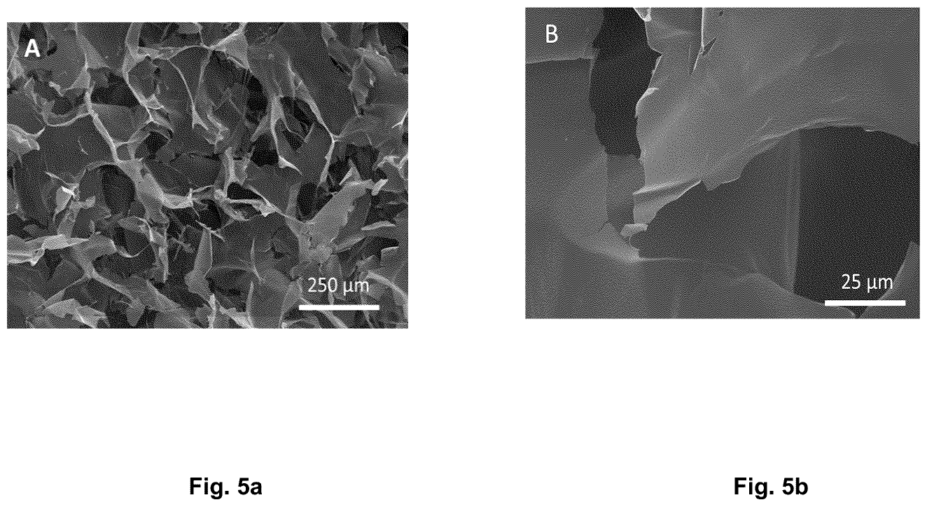

The morphology of chemical crosslinked cellulose aerogel was analysed with scanning electron microscopy (SEM) spectroscopy, as illustrated in FIGS. 5a and 5b, representing SEM pictures of the surface of a nanocellulose aerogel pad according to the invention, at two different magnifications, respectively with a 250 μm scale and with a 25 μm scale. It appears from FIGS. 5a and 5b that the obtained chemically crosslinked CNF aerogel exhibits an interconnected porous structure with a pore size of aerogel between 100 and 200 μm. The microporous structure of CNF aerogel is formed during the freeze-drying process, while the water in the nanocellulose hydrogel turned into ice crystals which was followed by subsequent sublimation forming voids in the nanocellulose aerogel. Since the pore size of CNF aerogel is 2-folders bigger than that of the glass fibre based conjugate pad ($1 μm), the capillary effect offered by CNF aerogel is decreased which can delay the flow of the sample during the test.

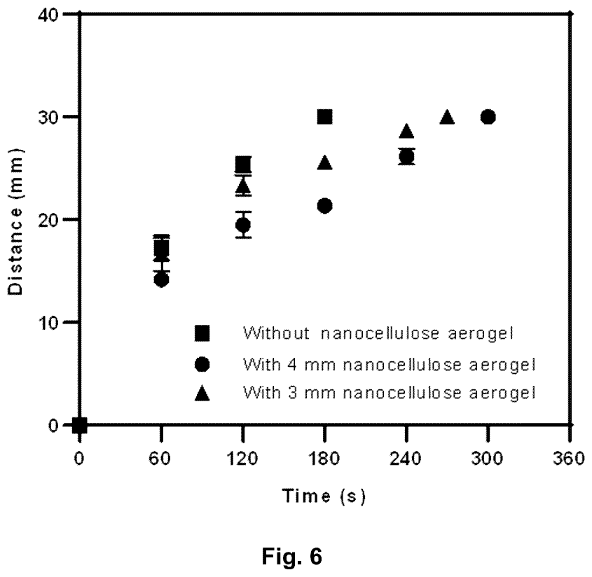

Migration distances as a function of time with and without the nanocellulose aerogel have been examined, as illustrated in FIG. 6 that represents a schematic diagram of the analytical sensitivity improvement of LFIA by different lengths of nanocellulose aerogel and migration time with and without the nanocellulose aerogel pad vs wicking distance on the nitrocellulose membrane.

It appears from FIG. 6 that the time of wicking distance on the nitrocellulose membrane increased for different lengths of nanocellulose aerogel pad by 90 seconds for 3 mm and by 120 seconds for 4 mm, respectively. Therefore, it shows that the use of nanocellulose aerogel can increase the migration time by at least 60 seconds, preferably by more than 90 seconds, even more preferably by at least 120 seconds.

As a consequence, the lateral flow strip, when assisted with the nanocellulose aerogel, gives a 100-fold LFIA analytical sensitivity improvement in the detection of mouse IgG with IgG-Anti-IgG model system (0.1 ng/ml) in comparison to the conventional LFIA, and linear range from 0.1 ng/ml to 100 ng/ml.

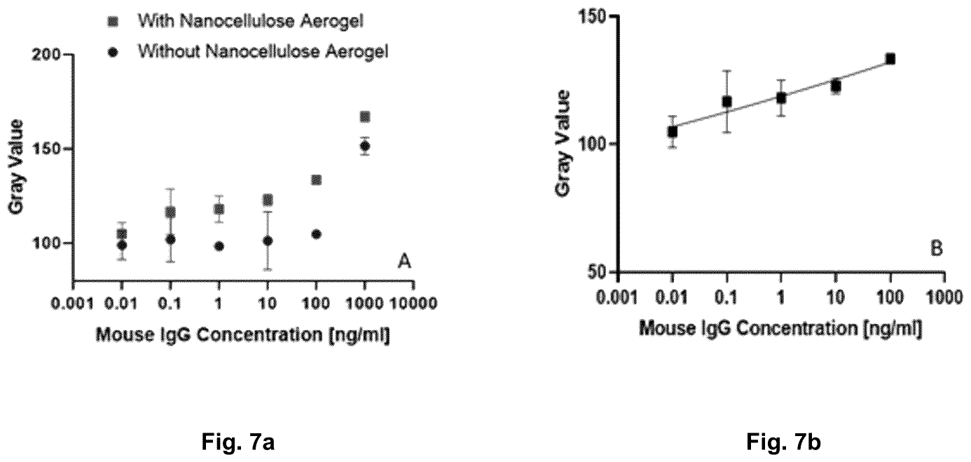

As illustrated in FIGS. 7a and 7b, calibration curves were made by plotting the obtained value of grayscale pixel of the test line against different concentrations of mouse IgG with a fitting curve in a log-log scale.

FIG. 7a illustrates the calibration curve of mouse IgG obtained by conventional LFIA and LFIA with nanocellulose aerogel, while FIG. 7b illustrates the linear range of mouse IgG obtained by nanocellulose assisted LFIA, a sensitivity of detection is 0.1 ng/ml and a linear range was achieved from 0.1 ng/ml to 100 ng/ml (R=0.9696, Y=2.08+0.023 log X).

It appears from the preceding description that the nanocellulose aerogel LFIA device according to the present invention offers better sensitivity and dynamic range with respect to conventional LFIA devices, while being still easy to manufacture on a large scale, and being a practical solution for point-of-care assessments.

Of course, the present description discloses a specific application regarding the predefined entity to be detected and the corresponding free labelled entity and detection entities, but the one skilled in the art will encounter no particular difficulty to adapt the present teaching to his needs by implementing different entities systems in connection with the structure of the nanocellulose aerogel LFIA device according to the invention, without going beyond the scope of the latter as defined by the appended claims. In a similar way, the dimensions that are mentioned in the present disclosure, as well as the specific details of the fabrication process, are provided as non-limiting examples and must not be considered as limiting the scope of the invention.

1-16. (canceled)

17. A method for the fabrication of a lateral flow test device intended to detect the presence of at least one predefined chemical, biological or biochemical entity in a sample, comprising the steps consisting in providing, following the flow direction of the sample on the test device:
a sample pad,
a conjugate pad intended to include at least one free labelled entity, which is optically or magnetically detectable and is adapted to react exclusively with said at least one predefined chemical, biological or biochemical entity so as to create a combined entity, and
a working membrane including at least one test line intended to bear:
either a first type detection entity adapted to react exclusively with said combined entity in order to immobilize said combined entity on said working membrane,
or a second type detection entity adapted to react exclusively with said at least one free labelled entity in order to immobilize said at least one free labelled entity on said working membrane,
the method comprising a further step of integrating a fluid flow regulating pad in the lateral flow test device, wherein said fluid flow regulating pad includes a nanocellulose aerogel pad manufactured by implementing steps consisting in: a) providing a hydrogel containing nanocellulose fibers,
b) conducting a chemical crosslinking of said nanocellulose fibers,
c) conducting a lyophilisation of the hydrogel containing the crosslinked nanocellulose fibers so as to define a nanocellulose aerogel, and
d) compacting and shaping a predefined amount of said nanocellulose aerogel so as to define said nanocellulose aerogel pad.

18. The method of claim 17, wherein said nanocellulose fibers are carboxylic nanocellulose fibers (CNF) of the Tempo-CNF type.

19. The method of claim 18, wherein said step b) includes a preliminary operation consisting in stirring a hydrogel solution containing between 0.5 and 5% in weight of Tempo-CNF for 30 to 60 mins at a temperature comprised between 20 and 30° C.

20. The method of claim 19, wherein said hydrogel solution is stirred at a stirring rate comprised between 1000 and 3000 rpm.

21. The method of claim 19, wherein said step b) includes additional operations consisting in
adding between x/2000 and x/500 g of 1,2,3,4-Butanetetracarboxylic acid (BTCA) powder and between x/20000 and x/5000 g of sodium hydrosulphite (Na₂S₂O₄) powder to x g of said stirred Tempo-CNF hydrogel solution, and
stirring the corresponding solution during at least 6 hours at a temperature comprised between 20 and 30° C.

22. The method of claim 17, wherein said step c) includes operations consisting in
pouring said hydrogel solution containing crosslinked nanocellulose fibers in a container such that said hydrogel solution has a final height in said container comprised between 0.5 and 10 mm,
keeping said container at a temperature comprised between 10 and 30° C. for 10 to 60 mins,
storing said container at a temperature comprised between −30 and −10° C. during at least 6 hours, and
freeze drying said hydrogel solution containing crosslinked nanocellulose fibers at a temperature comprised between −65 and −50° C. during at least 20 hours by lyophilizing.

23. The method of claim 18, wherein said step c) includes operations consisting in
pouring said hydrogel solution containing crosslinked carboxylic nanocellulose fibers in a container such that said hydrogel solution has a final height in said container comprised between 0.5 and 10 mm,
keeping said container at a temperature comprised between 10 and 30° C. for 10 to 60 mins,
storing said container at a temperature comprised between −30 and −10° C. during at least 6 hours, and
freeze drying said hydrogel solution containing crosslinked carboxylic nanocellulose fibers at a temperature comprised between −65 and −50° C. during at least 20 hours by lyophilizing.

24. The method of claim 17, wherein said step d) consists in applying a weight comprised between 0.5 and 10 kg on said nanocellulose aerogel during at least 10 mins to shape a nanocellulose aerogel pad having a thickness approximately comprised between 0.1 and 2 mm.

25. The method of claim 18, wherein said step d) consists in applying a weight comprised between 0.5 and 10 kg on said nanocellulose aerogel during at least 10 mins to shape a nanocellulose aerogel pad having a thickness approximately comprised between 0.1 and 2 mm.

26. The method of claim 17, further including an operation of passivation of at least part of the surface of said nanocellulose aerogel pad.

27. The method of claim 18, further including an operation of passivation of at least part of the surface of said nanocellulose aerogel pad.

28. The method of claim 17, wherein said nanocellulose aerogel pad is arranged, in the lateral flow test device, so as to contact said conjugate pad and said working membrane.

29. The method of claim 18, wherein said nanocellulose aerogel pad is arranged, in the lateral flow test device, so as to contact said conjugate pad and said working membrane.

30. A lateral flow test device intended to detect the presence of at least one predefined chemical, biological or biochemical entity in a sample, comprising, following the flow direction of the sample on the test device,
a sample pad,
a conjugate pad intended to include at least one free labelled entity, which is optically or magnetically detectable and is adapted to react exclusively with said at least one predefined chemical, biological or biochemical entity so as to create a combined entity, and
a working membrane including at least one test line intended to bear:
either a first type detection entity adapted to react exclusively with said combined entity in order to immobilize said combined entity on said working membrane,
or a second type detection entity adapted to react exclusively with said at least one free labelled entity in order to immobilize said at least one free labelled entity on said working membrane,
the test device further including a nanocellulose aerogel pad made of a compacted aerogel containing crosslinked nanocellulose fibers.

31. The device of claim 30, wherein said nanocellulose aerogel pad is arranged so as to contact said conjugate pad and said working membrane.

32. The device of claim 30, wherein said nanocellulose aerogel pad has a thickness comprised between 0.1 and 2 mm and a length comprised between 1 and 8 mm.

33. The device of claim 31, wherein said nanocellulose aerogel pad has a thickness comprised between 0.1 and 2 mm and a length comprised between 1 and 8 mm.

34. The device of claim 30, wherein the device is configured to detect at least one predefined antibody, said free labelled entity comprising a first anti-antibody adapted to react with said at least one predefined antibody to create said combined entity.

35. The device of claim 31, wherein the device is configured to detect at least one predefined antibody, said free labelled entity comprising a first anti-antibody adapted to react with said at least one predefined antibody to create said combined entity.

36. The device of claim 30, wherein said at least one free labelled entity contains one or more of the entities belonging to the group consisting in gold, a latex, a fluorophore, a ferromagnetic or paramagnetic entity.

37. The device of claim 31, wherein said at least one free labelled entity contains one or more of the entities belonging to the group consisting in gold, a latex, a fluorophore, a ferromagnetic or paramagnetic entity.

38. The device of claim 30, wherein
said sample pad is made of cellulose fiber,
said conjugate pad is made of glass fiber, and
said working membrane is made of nitrocellulose.

39. The device of claim 31, wherein
said sample pad is made of cellulose fiber,
said conjugate pad is made of glass fiber, and said working membrane is made of nitrocellulose.
